(19) United States
(12) Patent Application Publication
(10) Pub. No.: US 2024/0076865 A1
(43) Pub. Date: Mar. 7, 2024

(54) URINAL WITH BUILT-IN URINE DETECTION

(71) Applicant: Taiwan RedEye Biomedical Inc., Hsinchu City (TW)

(72) Inventors: Tsung-Jui LIN, Hsinchu City (TW); Shuo-Ting YAN, Hsinchu City (TW)

(73) Assignee: Taiwan RedEye Biomedical Inc., Hsinchu City (TW)

(21) Appl. No.: 17/903,338

(22) Filed: Sep. 6, 2022

(51) Int. Cl. E03D 13/00; A61B 5/00; A61B 5/20; G01N 33/50

(52) U.S. Cl. CPC E03D 13/005; A61B 5/0082; A61B 5/207; A61B 5/7445; G01N 33/50; A61B 2560/0462

(57) ABSTRACT

A urinal with built-in urine detection, comprises a urinal body, a detector, a processor and a display, the urinal body is disposed with a urine slot, and a urine drainage hole is disposed on a bottom of the urine slot; the detector is arranged in the urine slot and includes a casing, a detection channel for urine to pass through, a light source module and a sensing module. The light source module emits a detection beam into the detection channel, and the sensing module receives the detection beam passing through the urine, and generates a light intensity signal according to the detection beam; the processor electrically connects to the light source module and receives the light intensity signal, and calculates contents of various components in the urine according to the light intensity signal, to generate a test result and displays the test result in a display screen.

### BACKGROUND OF THE INVENTION

### 1. Field of the Invention

The present invention relates to a kind of toilet or urinal, in particular to a urinal with built-in urine detection.

### 2. Description of the Related Art

In order to monitor health conditions, the general public will regularly take health examinations. Health examinations usually require blood or urine tests such as urinalysis and other physiological tests, and patients with past medical histories especially need frequent blood and urine tests regarding values or indexes of various contents therein to continuously track the body conditions and to reduce chances of occurrences of symptoms. For example, uric acid is a final product of metabolism after a human body decomposes cellular nucleic acid (also known as purine). When a human body ingests too much food with high purine contents, or when a human body produces too much uric acid, or when kidneys have poor ability to process uric acid, symptoms such as hyperuricemia, gouty arthritis, kidney stones, joint deformities, etc. tend to occur. Hence it is necessary to measure the uric acid content in the urine through urinalysis to check whether there is any health threat.

Most of the present urinalyses utilize optical reagents and photoelectric analysis for detection, hence a test subject needs to go to a professional medical institution for the urinalysis. Usually results of a urinalysis are not disclosed to the test subject until several days afterwards. In addition, the test subject needs to put their urine first into a designated container such as a paper cup, pours it into a test tube, and then delivers the test tube to a personnel in charge for subsequent processing. Obviously, the aforementioned operation flow is time-consuming and inconvenient.

It can be seen that the present urinalysis lacks immediacy, and the convenience also needs to be enhanced.

### SUMMARY OF THE INVENTION

In view of the above-mentioned, a urinal with built-in urine detection of the present invention can enhance the convenience and efficiency of urine detection.

In order to achieve the foregoing purpose, the urinal with built-in urine detection of the present invention includes:
a urinal body, having a urine slot; wherein a urine drainage hole is disposed on a bottom surface of the urine slot;
a detector, disposed in the urine slot, including:
   a casing;
   a detection channel, for urine to pass through;
   a light source module, disposed in the casing, to emit a detection beam into the detection channel; and
   a sensing module, disposed in the casing, to receive the detection beam penetrating through the urine, and to generate a light intensity signal according to the detection beam received;
a processor, electrically connected to the light source module and the sensing module, wherein the processor receives the light intensity signal, and calculates contents of various components in the urine according to the light intensity signal and thereby generates a test result; and
a display, electrically connected to the processor, to receive the test result and to display the test result in a display screen.

The urinal with built-in urine detection of the present invention carries out urinalysis by a detector disposed in a urine slot, and as long as the test subject urinates normally and allows urine to flow into the urine slot, then urinalysis can be carried out directly. It is no longer necessary to use containers such as test tubes or paper cups to hold urine for urinalysis, which effectively improves the convenience of urinalysis. The present invention can analyze the urine to get test result in real time, and display the test result in a display screen for a user to understand the test result, which further reduces waiting time for test results compared with that of a conventional urinalysis.

### BRIEF DESCRIPTION OF THE DRAWINGS

FIG. 1 shows a perspective view of the urinal with built-in urine detection of the present invention;

FIG. 2 shows a partial sectional view of the urinal with built-in urine detection of the present invention;

FIG. 3 shows a cross-sectional view of the urinal with built-in urine detection of the present invention;

FIG. 4 shows an operational view of the urinal with built-in urine detection of the present invention;

FIG. 5 shows a block schematic diagram of the urinal with built-in urine detection of the present invention;

FIG. 6 shows an operational flow diagram of the urinal with built-in urine detection of the present invention;

FIG. 7 shows light absorbance spectra of various substances in urine; and

FIG. 8 shows a light absorbance spectrum of uric acid.

### DETAILED DESCRIPTION OF THE INVENTION

In the following, the technical solutions in the embodiments of the present invention will be clearly and fully described with reference to the drawings in the embodiments of the present invention. Obviously, the described embodiments are only a part of, not all of, the embodiments of the present invention. Based on the embodiments of the present invention, all other embodiments obtained by a person of ordinary skill in the art without creative efforts shall fall within the protection scope of the present invention.

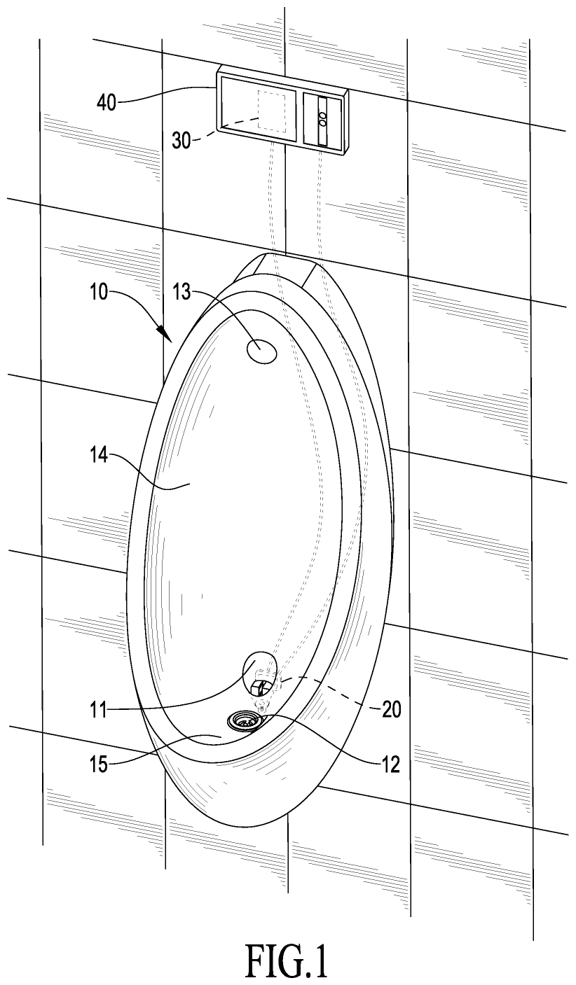

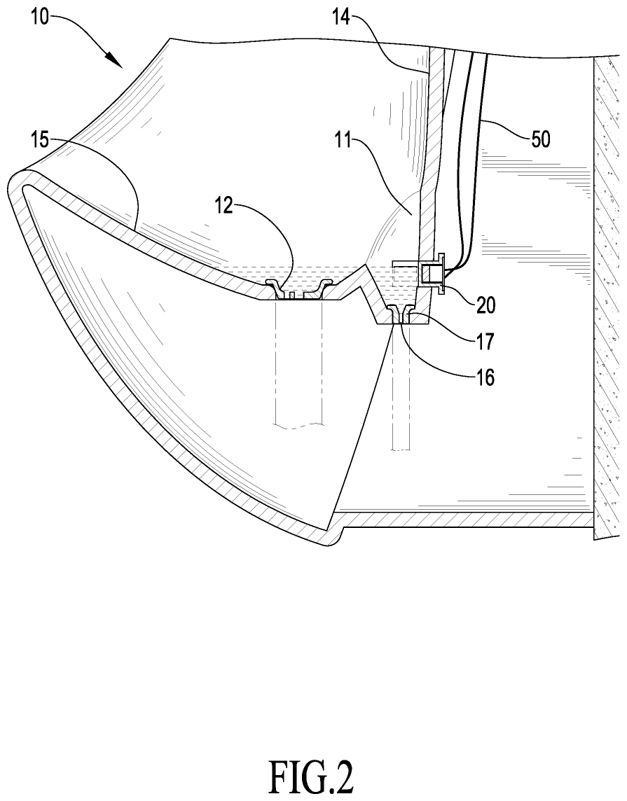

Please refer to FIGS. 1 and 2: the urinal with built-in urine detection of the present invention includes a urinal body 10, a detector 20, a processor 30 and a display 40. A toilet for defecation can also be adopted to substitute the urinal body 10. In the embodiments of FIGS. 1 to 3, the urinal body 10 is taken as an example. The urinal body 10 has a urine slot 11, a drainage hole 12 and a flush sensor 13, and the urinal body 10 has a sidewall 14, the sidewall 14 is recessed to form a pool 15, the urine slot 11 is disposed on the sidewall 14 of the urinal body 10, and a urine drainage hole 16 is disposed on a bottom surface of the urine slot 11, and the urine drainage hole 16 of the urine slot 11 is connected to a drain pipe (not shown in the figures) of the urinal body 10. A funnel piece 17 is disposed in the urine drainage hole 16. An opening end of the funnel piece 17 on the sidewall 14 of the urinal body 10 is wider than another opening end of the funnel piece 17 connected to the drain pipe. When urine enters the urine slot 11 and is to be discharged from the urine drainage hole 16, a funnel-shaped design of the funnel piece 17 reduces the speed at which the urine is discharged from the urine slot 11 to the drain pipe, so as to facilitate a urinalysis in the urine slot 11. The drainage hole 12 is connected to the drain pipe of the urinal body 10. The flush sensor 13 is used to sense objects and drive the urinal body 10 to flush the sidewall 14. The flush sensor 13 is installed on the sidewall 14 of the urinal body 10. When the user approaches or leaves the urinal body 10, as the sensing value of the flush sensor 13 is thereby changed, the urinal body 10 is driven by the flush sensor 13 to flush to clean the sidewall 14.

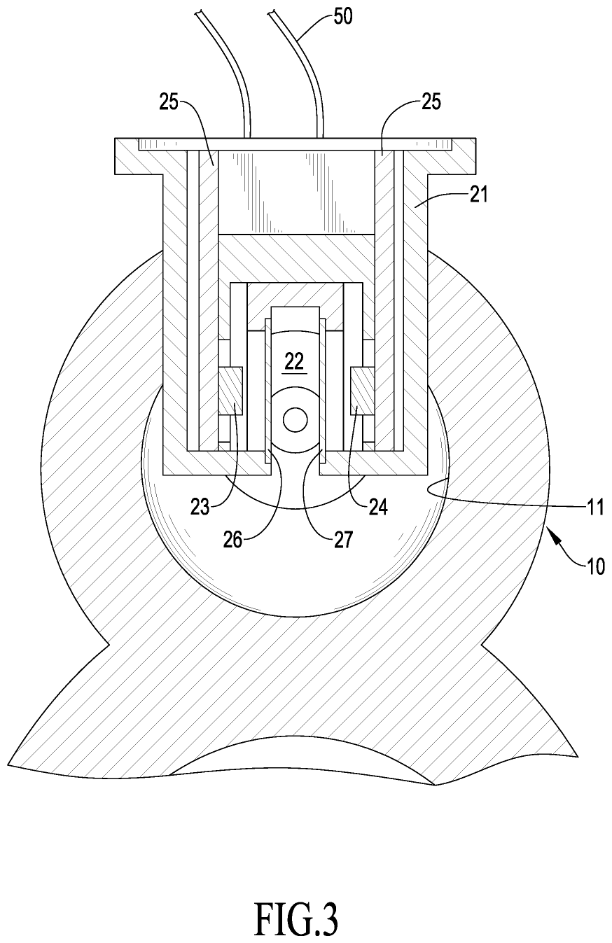

Please refer to FIG. 3: the detector 20 is disposed in the urine slot 11. The detector 20 includes a casing 21, a detection channel 22, a light source module 23, and a sensing module 24; the casing 21 is waterproof. The light source module 23 and the sensing module 24 are disposed in an inner space of the casing 21, and the light source module 23 and the sensing module 24 are covered by the casing 21 to prevent urine from infiltrating. When the urine slot 11 is filled with urine, the detection channel 22 allows the urine to pass through. The light source module 23 and the sensing module 24 are disposed on opposite sides of the detection channel 22 and also disposed on at least one circuit board 25 which is disposed in the inner space of the casing 21. A first transparent lens 26 and a second transparent lens 27 of the detector 20 are respectively disposed on the opposite sides of the detection channel 22. The first transparent lens 26 is arranged between the light source module 23 and the detection channel 22. The second transparent lens 27 is arranged between the sensing module 24 and the detection channel 22. Among them, the casing 21 can be transparent, which does not affect the functions of the first transparent lens 26 and the second transparent lens 27.

The light source module 23 is configured to emit a detection beam into the detection channel 22. The detection beam sequentially penetrates the first transparent lens 26, the urine in the detection channel 22 and the second transparent lens 27 and then passes through the sensing module 24. The sensing module 24 generates a light intensity signal according to the received detection beam, wherein the light source module 23 can be a mercury lamp, a gas lamp, a laser light source, an LED lamp or other light-emitting devices. The sensing module 24 can be a photosensitive element such as a photo sensor, a photodiode array (PDA) sensor, a spectrometer, a complementary metal oxide semiconductor (CMOS) sensor, etc., and the light intensity signal is expressed in terms of light absorbance. The detection beam can contain light of different wavelengths according to the different components to be detected, and the present invention can measure the pH value of urine, urine sugar, urine protein, occult blood, uric acid, nitrite, ketone bodies and other parameters.

According to the formula of the Beer-Lambert law:

$$A = α Lc and A = - log \frac{I}{{I}_{0}} ;$$

Wherein, A is the light absorbance, α is the light absorbance coefficient, L is the optical path length, c is the concentration, I₀ is the incident light intensity, and I is the transmitted light intensity.

When light enters an object, light-absorbing materials in the object can absorb part of the light energy, so that the light intensity of the light transmitted out of the object is attenuated, and the absorbed energy can be expressed in terms of light absorbance A. The energy difference of the incident light intensity I₀ and the transmitted light intensity I is used to calculate the light absorbance A of the object. When the detector 20 performs urinalysis in the urine, the detection beam emitted by the light source module 23 passes through the urine in the detection channel 22 and is received by the sensing module 24, and part of the light energy of the detection beam is absorbed by the urine. Therefore, the sensing module 24 can generate a light intensity signal from the light intensity of the detection beam. The light intensity signal is expressed in terms of light absorbance, and the concentration of various components in the urine can be calculated based on the light absorbance. In addition to performing urinalysis with optical means of the present invention, the detector 20 of the present invention can also perform urinalysis utilizing the principles of biochemical method, electrochemical method, conductivity analysis method, etc.

The processor 30 is electrically connected to the flush sensor 13, the light source module 23 and the sensing module 24. The processor 30 controls the operation of the flush sensor 13, the light source module 23 and the sensing module 24, and receives the light intensity signal output from the sensing module 24. The processor 30 calculates contents of various components in the detected urine based on the light intensity signal to generate a test result which is transmitted to the display 40, wherein the processor 30 can be a central processor or a microcontroller.

The display 40 is electrically connected to the processor 30, and receives the test result transmitted by the processor 30. The display 40 can present the test result in a display screen in term of numerical values or images for the user to view. In this embodiment, the display 40 can be installed separately from the urinal body 10. In FIG. 1, the display 40 and the urinal body 10 are installed on the same wall, and the display 40 can be installed at the same height as the test subject's sight when using the urinal body 10, where it is convenient for the test subject to check the test result through the display screen. The processor 30 can be disposed on the back of the display 40 and is electrically connected to the detector 20 through an electric wire 50.

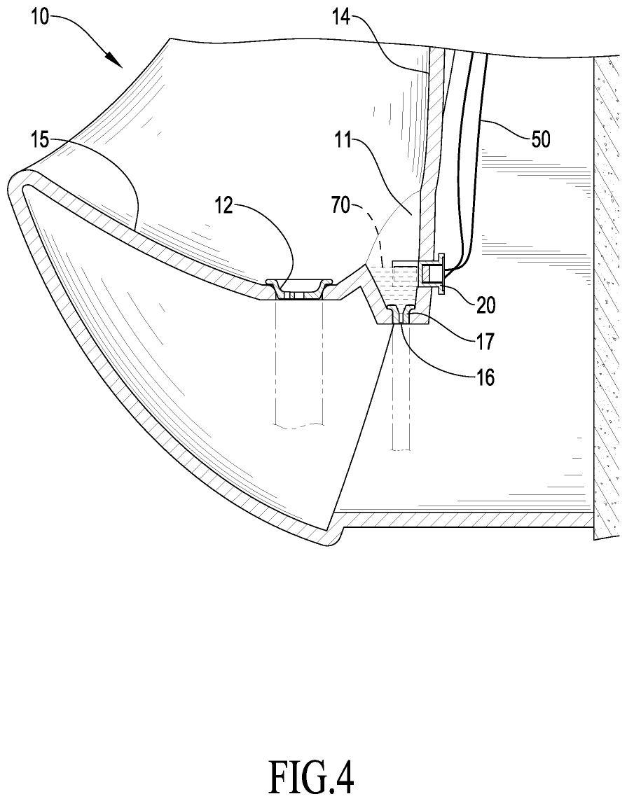

In addition, please refer to FIG. 1 and FIG. 2, the flush sensor 13 is located above the drainage hole 12, and the urine slot 11 is formed on the sidewall 14, and the urine slot 11 is disposed between the flush sensor 13 and the drainage hole 12 and on a line connecting the flush sensor 13 and the drainage hole 12. Further referring to FIG. 4, as a user urinates, the urine slot 11 receives urine 70, and the funnel-shaped design of the funnel piece 17 slows down the flow rate of the urine 70, so that the urine 70 accumulates in the urine slot 11, and when the liquid level of the urine 70 covers the detector 20, the urine 70 passes through the detection channel 22 of the detector 20. At this moment, the detector 20 performs urinalysis, the light source module 23 emits a detection beam, and the detection beam passing through the urine 70 in the detection channel 22 is received by the sensing module 24 which generates a light intensity signal according to the received light intensity of the detection beam.

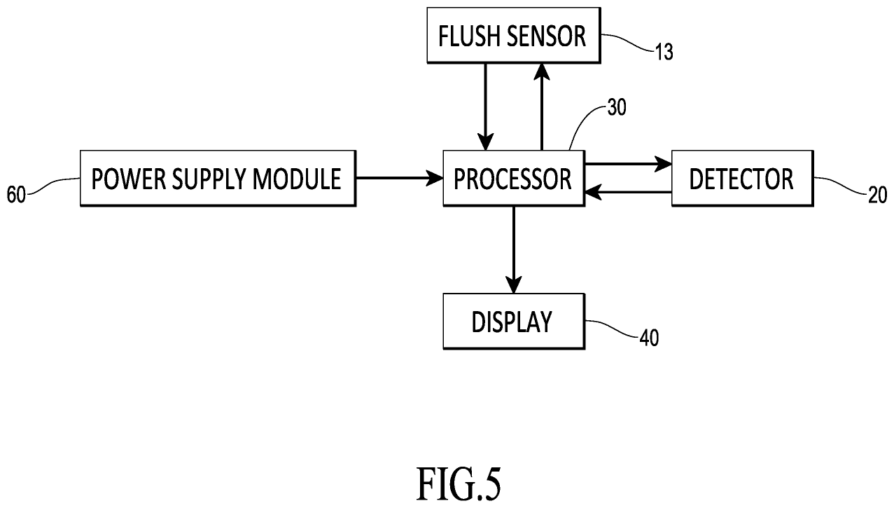

Please refer to FIG. 5: the urinal with built-in urine detection of the present invention includes a power supply module 60. The power supply module 60 is electrically connected to the processor 30 and supplies power to the processor 30. The power supply module 60 can be an adapter, a transformer or a converter to receive power from utility, an AC power source or a DC power source, and to output a DC power or an AC power to supply power to the processor 30. Or the power supply module 60 is a DC power source or an AC power source, such as a battery, which can directly output power to the processor 30.

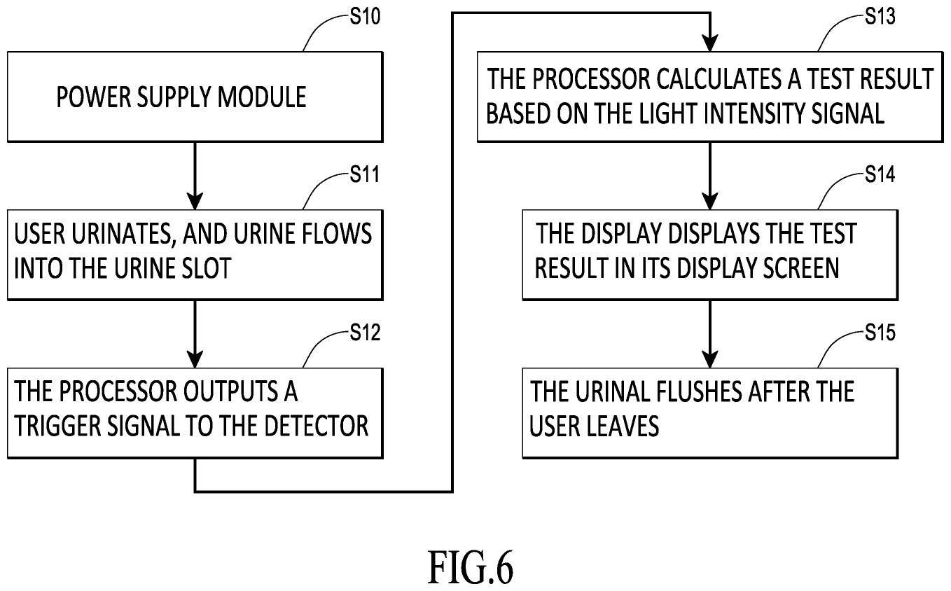

Further referring to FIG. 6, an operation flow of the urinal with built-in urine detection of the present invention is as follows.

S10: Detecting that a user is approaching, the flush sensor 13 drives the urinal body 10 to flush water.

S11: The user urinates, and the urine flows into the urine slot 11. When the urine flows to the urine slot 11, the water accumulating during flushing is replaced by the user's urine.

S12: After the flush sensor 13 detects the user's approach and drives the urinal to flush, the processor 30 outputs a trigger signal to the detector 20, and according to the trigger signal, the processor 30 controls the light source module 23 to emit a detection beam. When the detection beam penetrates the urine in the detection channel 22 and is received by the sensing module 24, the sensing module 24 generates a light intensity signal according to the received light intensity of the detection beam, and transmits the light intensity signal to the processor 30.

S13: After receiving the light intensity signal, the processor 30 calculates the detected contents of various components in the urine based on the light intensity signal and generates a test result, and transmits the test result to the display 40.

S14: The display 40 displays the test result in its display screen.

S15: After the user leaves, the flush sensor 13 drives the urinal body 10 to flush. The residual urine is taken away by the water flow to achieve cleaning effects.

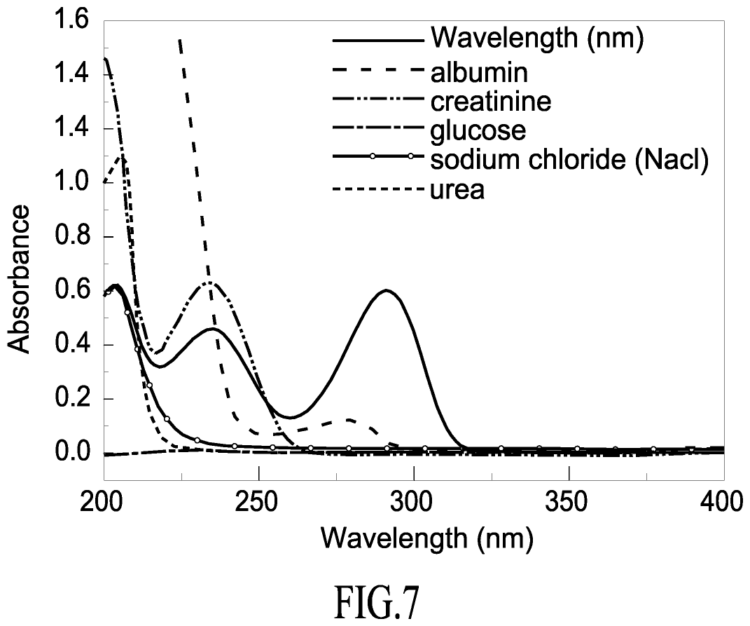

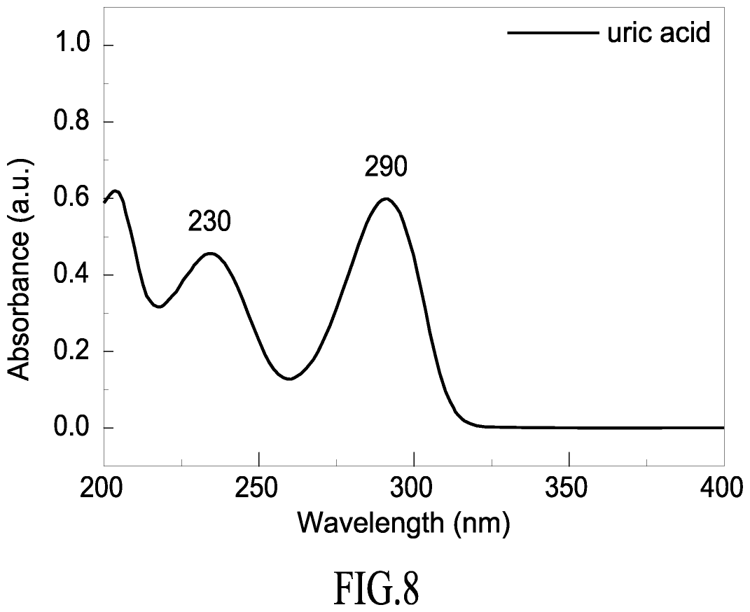

Please refer to FIG. 7, which is a light absorbance spectrum diagram of various substances in urine. From FIG. 7, it can be found that the light absorbance peaks of uric acid, albumin, creatinine, glucose, salt and urea in urine are different. Further referring to FIG. 8, taking uric acid as an example, the light absorbance peak of uric acid does not overlap with the light absorbance peaks of other substances, and the wavelength of the light absorbance peak of uric acid solution is between 275 nm and 315 nm. In other words, as the detection beam with various wavelengths penetrates a urine sample, in contrast to the light absorbance for other wavelengths, the light absorbance for wavelengths between 275 nm and 315 nm is prominent and the light absorbance phenomenon of other substances in the urine is not significant. Therefore, if the detection beam with a wavelength of 275 nm to 315 nm is used for uric acid detection, the accuracy will not be affected by the interference of other substances in the urine. Similarly, when various components in urine are to be detected, the wavelength of the detection beam can be adjusted, or various wavelengths of light can be added to the detection beam to detect the light intensity of the light with various wavelengths after passing through the urine.

In sum, a urine slot 11 is disposed in a urinal body 10 of the urinal with built-in urine detection of the present invention, and a urine detection is carried out by a detector 20 in the urine slot 11, and a test result is immediately displayed to a user. Compared with conventional technologies, the test subject does not need to store urine in a container, and the test is performed when the user urinates in the urinal, which improves the convenience of urinalysis. Accordingly, the test subject does not need to wait for hours or days for a test report, and is informed about their physical conditions in real time.

The aforementioned are preferred embodiments of the present invention. It should be noted that for those of ordinary skill in the art, without departing from the principles of the present invention, certain improvements and retouches of the present invention can still be made, which are nevertheless considered as within the protection scope of the present invention.

What is claimed is:

1. A urinal with built-in urine detection, including:
a urinal body, having a urine slot; wherein a urine drainage hole is disposed on a bottom surface of the urine slot; a detector, disposed in the urine slot, including:
a casing;
a detection channel, for urine to pass through;
a light source module, disposed in the casing, to emit a detection beam into the detection channel; and
a sensing module, disposed in the casing, to receive the detection beam penetrating through the urine, and to generate a light intensity signal according to the detection beam received;
a processor, electrically connected to the light source module and the sensing module, wherein the processor receives the light intensity signal, and calculates contents of various components in the urine according to the light intensity signal and thereby generates a test result; and
a display, electrically connected to the processor, to receive the test result and to display the test result in a display screen.

2. The urinal with built-in urine detection as claimed in claim 1, wherein a funnel piece is disposed in the urine drainage hole of the urine slot.

3. The urinal with built-in urine detection as claimed in claim 1, wherein the urinal body, the processor and the display are separately disposed.

4. The urinal with built-in urine detection as claimed in claim 1, wherein the light source module and the sensing module are disposed on opposite sides of the detection channel.

5. The urinal with built-in urine detection as claimed in claim 1, wherein the urinal body is disposed with a flush sensor, and when a user approaches or leaves the urinal body, the flush sensor drives the urinal body to flush.

6. The urinal with built-in urine detection as claimed in claim 5, wherein when the flush sensor detects the user's approach and drives the urinal body to flush, the processor outputs a trigger signal to the detector, and the light source module is controlled by the trigger signal to emit the detection beam.

7. The urinal with built-in urine detection as claimed in claim 1, the detector including:
a first transparent lens, disposed between the light source module and the detection channel; and
a second transparent lens, disposed between the sensing module and the detection channel;
wherein, the detection beam is received by the sensing module after sequentially passing through the first transparent lens, the urine in the detection channel and the second transparent lens.

8. The urinal with built-in urine detection as claimed in claim 1, the urinal body having a sidewall;
a pool, concavely formed in the sidewall, and a drainage hole formed at a bottom of the pool;
a flush sensor disposed on the sidewall and above the drainage hole; and
the urine slot formed on the sidewall and disposed on a line connecting the flush sensor and the drainage hole.
